(19) United States
(12) Patent Application Publication
(10) Pub. No.: US 2019/0191966 A1
(43) Pub. Date: Jun. 27, 2019

(54) INSERTION DEVICE

(71) Applicant: OLYMPUS CORPORATION, Tokyo (JP)

(72) Inventor: Kunihiko Hijihara, Tokyo (JP)

(73) Assignee: OLYMPUS CORPORATION, Tokyo (JP)

(21) Appl. No.: 16/286,862

(22) Filed: Feb. 27, 2019

Related U.S. Application Data
(63) Continuation of application No. PCT/JP2017/036138, filed on Oct. 4, 2017.

(30) Foreign Application Priority Data
Oct. 5, 2016 (JP) 2016-197504

(51) Int. Cl. A61B 1/005

(52) U.S. Cl. CPC A61B 1/0052

(57) ABSTRACT

An insertion device includes an insertion portion, a grasping portion, and a flexible tube that extends from a major angle side corresponding to a minor angle with respect to a center axis for an end portion on a proximal end side of the grasping portion, and the end portion is provided at a position where a thenar portion of an operator can come into contact with the grasping portion and the flexible tube when the operator grasps the grasping portion, and an extension direction of the flexible tube is inclined to the major angle side with respect to the center axis.

### CROSS REFERENCE TO RELATED APPLICATION

This application is a continuation application of PCT/JP2017/036138 filed on Oct. 4, 2017 and claims benefit of Japanese Application No. 2016-197504 filed in Japan on Oct. 5, 2016, the entire contents of which are incorporated herein by this reference.

### BACKGROUND OF THE INVENTION

### 1. Field of the Invention

The present invention relates to an insertion device including an insertion portion that has a longitudinal axis and is configured to be inserted into a subject, and a grasping portion that is provided to be connected to a proximal end in a direction along the longitudinal axis of the insertion portion.

### 2. Description of the Related Art

In recent years, insertion devices, for example, endoscopes, have been widely used in a medical field and an industrial field.

It is well known that an endoscope is configured to include an insertion portion which has a longitudinal axis and is configured to be inserted into a subject, an operation portion provided to be connected to a proximal end in a direction along the longitudinal axis of the insertion portion, a universal cord extending from the operation portion, and a connector that is provided at an extension end of the universal cord and is freely attachable and detachable to and from an external device.

The operation portion is provided with a bending operation member configured to bend a bending portion provided to the insertion portion, and various kinds of operation switches.

There is a well-known manipulation method in which an operator operates the bending operation member and the various kinds of operation switches provided to the operation portion by fingers of an operator's left hand while grasping the insertion portion by an operator's right hand and inserting the insertion portion into a subject in a state where the operation portion is grasped by the operator's left hand, for example.

However, with respect to an endoscope used in, for example, otolaryngology, an operator must perform an operation of inserting the insertion portion into a body cavity from a mouth or a nose of a sitting patient in a standing state. For this reason, it is sometimes necessary for the operator to bend the wrist of the operator's left hand grasping the operation portion at an unreasonable angle.

In view of such circumstances, an endoscope is well known which is capable of improving insertability of the insertion portion without bending the wrist of the hand grasping the operation portion at an unreasonable angle.

Japanese Patent Application Laid-Open Publication No. 2009-189685 discloses a configuration of an endoscope in which a grasping portion having a so-called gun-type (L-type) shape is provided to be connected to a proximal end in a longitudinal axis direction of an insertion portion, the grasping portion having an outer peripheral portion formed around a center axis intersecting a longitudinal axis at a minor angle and being provided with a bending operation member and various kinds of operation switches. A flexible tube configured to be connected to an external device extends from an end portion in a direction along the center axis of the grasping portion.

### SUMMARY OF THE INVENTION

An insertion device according to an aspect of the present invention comprises: an insertion portion that has a longitudinal axis and is configured to be inserted into a subject; a grasping portion that is formed around a center axis intersecting the longitudinal axis at a minor angle, provided to be connected to a proximal end in a direction along the longitudinal axis of the insertion portion, and is configured to be graspable by an operator; and a flexible tube extending from a side of a major angle corresponding to the minor angle with respect to the center axis for an end portion on a proximal end side in a direction along the center axis of the grasping portion, wherein the end portion is provided at a position where a thenar portion of the operator can come into contact with the grasping portion and the flexible tube when the operator grasps the grasping portion, and an extension direction of the flexible tube is inclined to the major angle side with respect to the center axis.

### BRIEF DESCRIPTION OF THE DRAWINGS

FIG. 1 is a diagram showing an endoscope according to a first embodiment;

FIG. 2 is a diagram showing a state in which an operator grasps an outer peripheral portion of a grasping portion of the endoscope of FIG. 1 by a left hand;

FIG. 3 is a partial cross-sectional view showing a cross section of the outer peripheral portion of the grasping portion taken along a line III-III in FIG. 1 together with the operator's left hand while the cross section of the outer peripheral portion is placed upside down;

FIG. 4 is a partial cross-sectional view showing the shape of a modification of the cross section of the outer peripheral portion of the grasping portion of FIG. 3 together with the operator's left hand; and

FIG. 5 is a diagram showing an endoscope according to a second embodiment.

### DETAILED DESCRIPTION OF THE PREFERRED EMBODIMENT(S)

Hereinafter, embodiments of the present invention will be described with reference to the drawings. Note that the drawings are schematic and the relationship of thicknesses and widths of respective members, the ratio of the thicknesses of the respective member, etc. are different from actual relationship, ratio, etc., and also needless to say, portions having dimensional relationships and ratios different among the drawings are included.

Note that in the following embodiments, an insertion device will be described by exemplifying an endoscope.

### First Embodiment

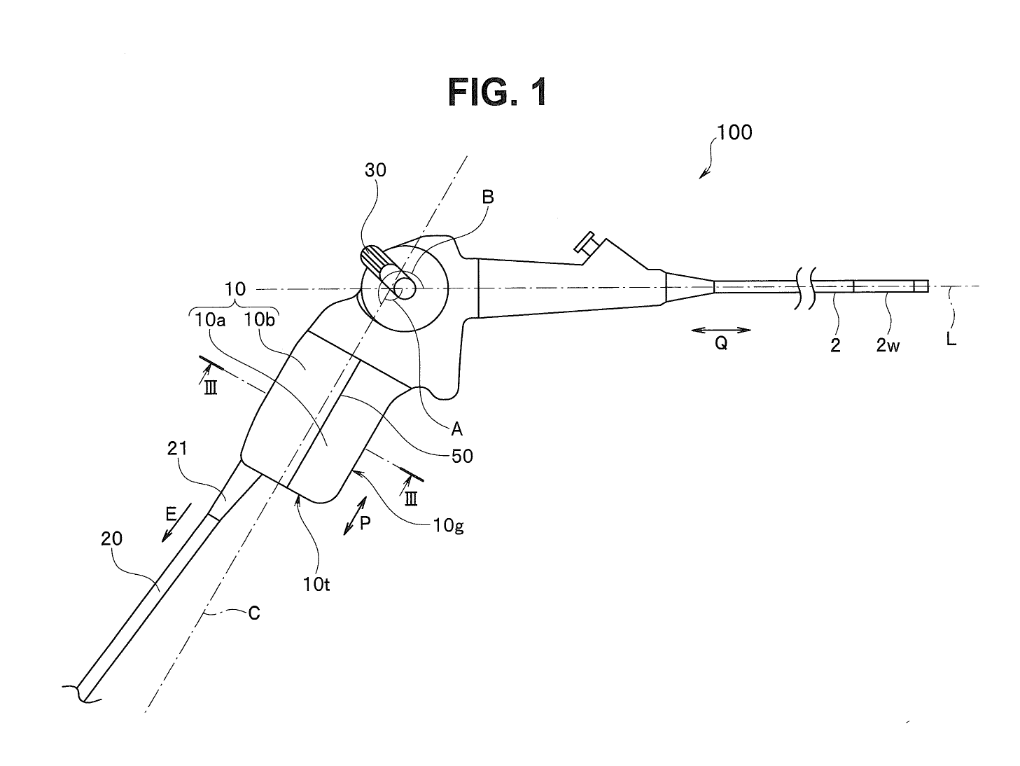

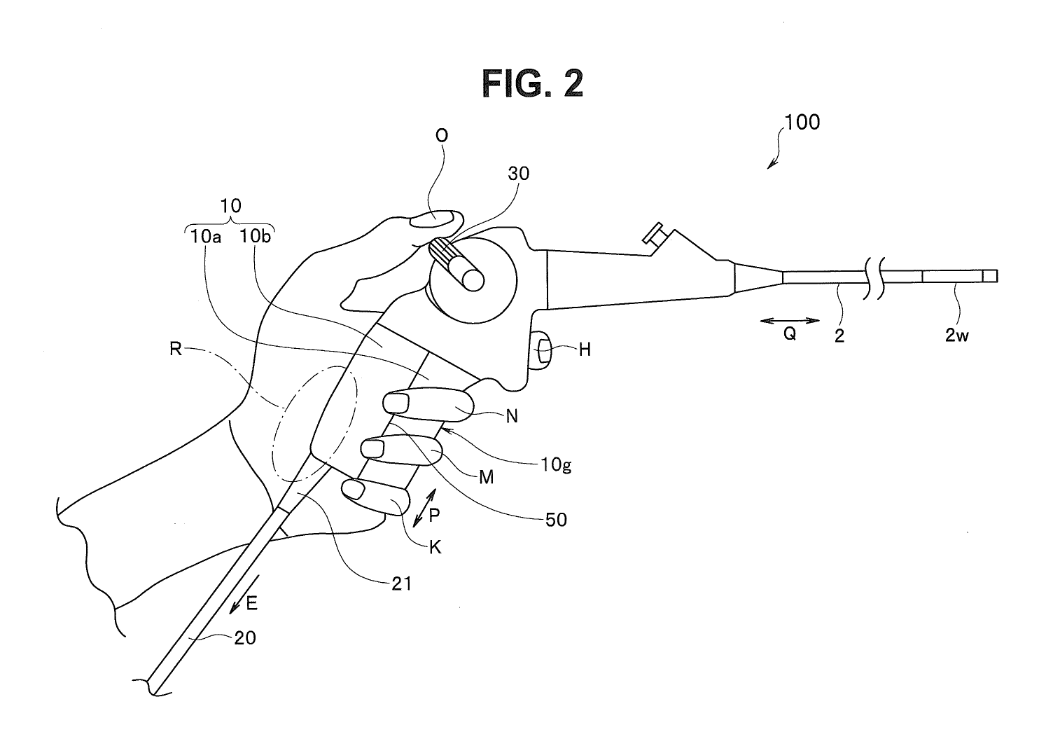

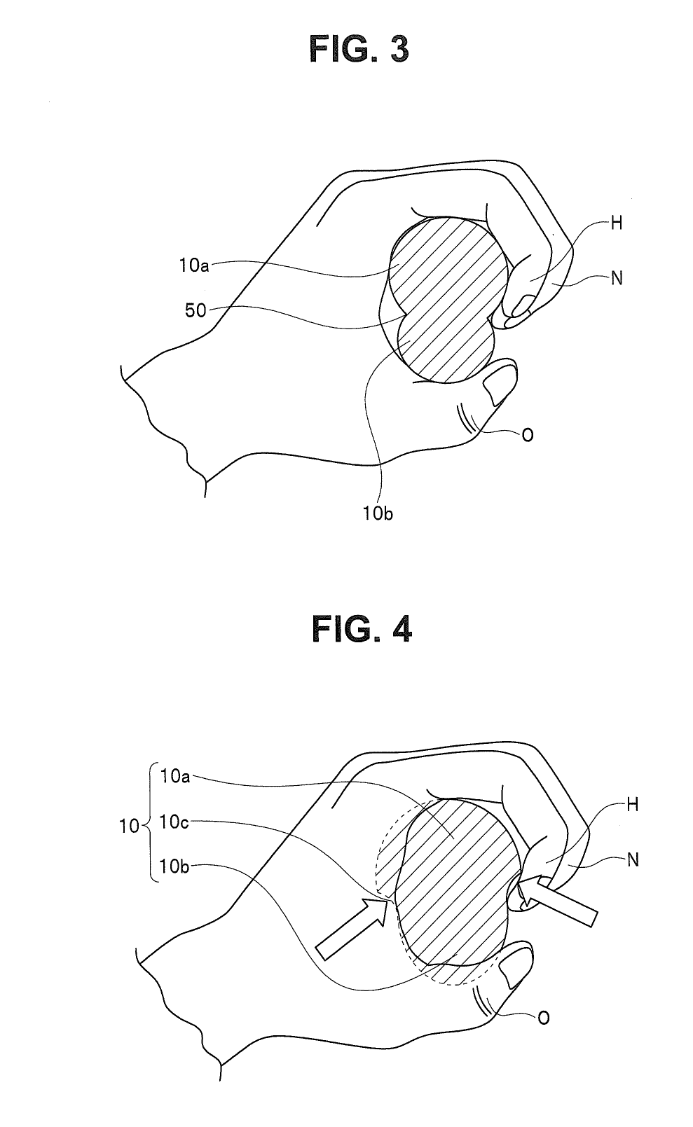

FIG. 1 is a diagram showing an endoscope according to the present embodiment, FIG. 2 is a diagram showing a state in which an operator grasps an outer peripheral portion of a grasping portion of the endoscope of FIG. 1 by the left hand, and FIG. 3 is a partial cross-sectional view showing a cross section of the outer peripheral portion of the grasping portion taken along a line III-III in FIG. 1 together with the operator's left hand while the cross section of the outer peripheral portion is placed upside down.

FIG. 4 is a partial cross-sectional view showing a shape of a modification of the cross section of the outer peripheral portion of the grasping portion of FIG. 3 together with the operator's left hand.

As shown in FIGS. 1 and 2, an endoscope 100 includes a main portion configured to include an insertion portion 2, a grasping portion 10, and a flexible tube 20.

The insertion portion 2 has a longitudinal axis L and is configured to be inserted into a subject, and also the insertion portion 2 has flexibility and is formed to be elongated. A bending portion 2w which is freely bendable in plural directions is provided on a distal end side in a direction Q along the longitudinal axis L.

The grasping portion 10 is provided to be connected to a proximal end of the insertion portion 2 in the direction Q. The grasping portion 10 has an outer peripheral portion 10g formed around a center axis C intersecting the longitudinal axis L at a minor angle A, an outer shape of which is substantially elliptical. In other words, the grasping portion 10 has a so-called gun-type shape.

Note that the minor angle A is set on an opposite side to a bending operation lever 30 described later as shown in FIG. 1. In other words, the outer peripheral portion 10g extends rearward and obliquely downward in FIGS. 1 and 2.

The grasping portion 10 is formed of resin or metal, and as shown in FIG. 3, the outer peripheral portion 10g may be formed to have a cross-sectional shape which is obtained by connecting a substantially semicircular large-diameter portion 10a and a small-diameter portion 10b and has two different radii.

In this case, the difference in radius between the large-diameter portion 10a and the small-diameter portion 10b causes a finger rest 50 to be formed along the center axis C at a connection portion between the large-diameter portion 10a and the small-diameter portion 10b as shown in FIG. 1.

As shown in FIGS. 2 and 3, the large-diameter portion 10a serves as a site with which pads of an index finger H, a middle finger N, a ring finger M, and a little finger K of the operator's left hand come into contact.

The small-diameter portion 10b serves as a site with which a thenar portion R of a thumb O and first joints of the index finger H, the middle finger N, the ring finger M, and the little finger K of the operator's left hand come into contact.

The finger rest 50 is configured to be capable of hooking the index finger H, the middle finger N, the ring finger M, and the little finger K of the operator's left hand.

Therefore, the outer peripheral portion 10g is formed in such a shape that not only the operator easily grasps the outer peripheral portion 10g of the grasping portion 10, but also the operator's left hand hardly becomes fatigued even when the operator grasps the outer peripheral portion 10g of the grasping portion 10 for a long time.

As shown in FIG. 4, the cross-sectional shape of the outer peripheral portion 10g may have a shape in which the large-diameter portion 10a and the small-diameter portion 10b are connected to each other by a circular arc 10c.

In this configuration, the pads of the index finger H, the middle finger N, the ring finger M, and the little finger K and the thenar portion R of the operator's left hand grasping the outer peripheral portion 10g come into contact with the circular arc 10c.

As shown in FIGS. 1 and 2, the bending operation lever 30 as an operation member configured to operate the bending portion 2w so as to bend the bending portion 2w is provided on a major angle B side corresponding to the minor angle A of the outer peripheral portion 10g of the grasping portion 10.

The bending operation lever 30 is provided at a position where the bending operation lever 30 can be operated by the thumb O of the operator's left hand grasping the outer peripheral portion 10g at the outer peripheral portion 10g.

The operation member configured to operate the bending portion 2w so as to bend the bending portion 2w is not limited to the bending operation lever, but may be a joystick or the like.

Although not shown in the drawing, various kinds of known operation switches are provided to the grasping portion 10, and these operation switches are provided at positions where the operation switches can be operated by the index finger H or the middle finger N of the operator's left hand grasping the outer peripheral portion 10g.

The flexible tube 20 is configured to connect the endoscope 100 to an external device, and extends from the major angle B side with respect to the center axis C at an end portion 10t in a direction P along the center axis C of the outer peripheral portion 10g.

More specifically, an extension direction E of the flexible tube 20 is substantially parallel to the center axis C or the extension direction E is inclined toward the major angle B side with respect to the center axis C.

As a result, as shown in FIG. 2, the flexible tube 20 comes into contact with the thenar portion R of the operator's left hand grasping the outer peripheral portion 10g.

Note that the flexible tube 20 more easily comes into contact with the thenar portion R when the extension direction E is inclined to the major angle B side with respect to the center axis C.

As shown in FIGS. 1 and 2, an end portion on the side of the outer peripheral portion 10g of the flexible tube 20 is covered with a bend preventing portion 21 as a cover member.

Note that the flexible tube 20 may be fixed in such a form that the bend preventing portion 21 is not provided, that is, the flexible tube 20 is exposed to the end portion on the side of the outer peripheral portion 10g.

Note that other components of the endoscope 100 are similar to the components of other endoscopes in which the grasping portion 10 has a gun-type shape, and thus description on these components will be omitted.

As described above, it is shown in the present embodiment that the flexible tube 20 extends from the major angle B side with respect to the center axis C for the end portion 10t.

According to the above configuration, even when the outer peripheral portion 10g is formed to be short in the direction P in order to make the endoscope 100 more compact and lighter, the flexible tube 20 surely comes into contact with the thenar portion R of the operator's left hand grasping the outer peripheral portion 10g.

Therefore, even when an operator has a large hand, the flexible tube 20 comes into contact with the thenar portion R, so that a contact area can be secured to be substantially equal to the contact area when the outer peripheral portion 10g is formed to be long in the direction P.

Therefore, the outer peripheral portion 10g is easy to grasp, and a sense of security can be given to the operator grasping the outer peripheral portion 10g.

In the case of medical endoscopes, for example, a twisting operation may be performed on the grasping portion 10.

Even in that case, since the flexible tube 20 extends from the major angle B side with respect to the center axis C for the end portion 10t, the flexible tube 20 approaches an operator's arm, so that the flexible tube 20 hardly disturbs during the twisting operation.

As described above, it is possible to provide the endoscope 100 having the grasping portion 10 that can be reduced in weight and size and enables stable grasping.

### Second Embodiment

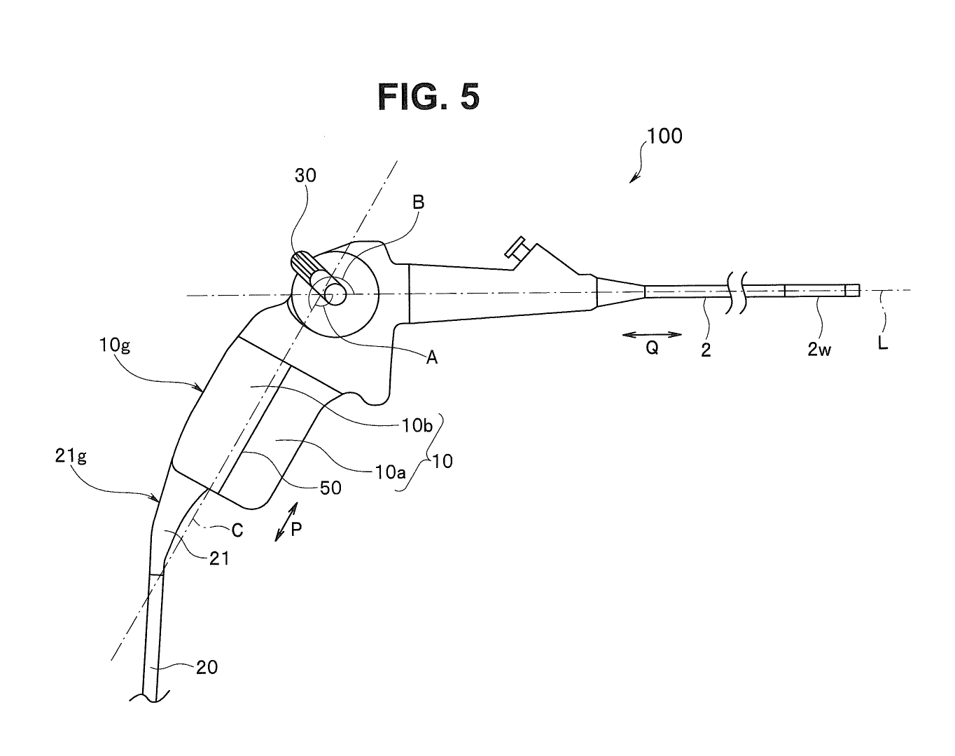

FIG. 5 is a view showing the endoscope of the present embodiment.

The configuration of the endoscope according to the second embodiment is different from the endoscope of the first embodiment shown in FIGS. 1 to 4 in that at least a part of the outer peripheral surface of the flexible tube is formed along an extension line on the major angle side of the outer peripheral portion of the grasping portion.

Therefore, only the difference will be described, and components similar to the component of the first embodiment are represented by the same reference signs, and description on these components will be omitted.

As shown in FIG. 5, in the present embodiment, at least a part of the outer peripheral surface 21g of the bend preventing portion 21 covering the flexible tube 20 is formed along an extension line on the side of the major angle B of the outer peripheral portion 10g. Note that other components are the same as the components of the first embodiment described above.

According to such a configuration, in addition to the effect of the above-described first embodiment, the contact area between the bend preventing portion 21 and the thenar portion R is larger than the contact area in the first embodiment, so that it is possible to grasp the grasping portion 10 more comfortably.

In the above-described first and second embodiments, the grasping portion 10 is shown to be grasped by the left hand, but is not limited to this style. The same effect can also be obtained in a case where the grasping portion 10 is grasped by the right hand.

The insertion device has been described by exemplifying the endoscope, but is not limited to the endoscope. Any insertion device is applicable insofar as the insertion device has a gun-type grasping portion.

What is claimed is:

1. An insertion device comprising:
an insertion portion that has a longitudinal axis and is configured to be inserted into a subject;
a grasping portion that is formed around a center axis intersecting the longitudinal axis at a minor angle, provided to be connected to a proximal end in a direction along the longitudinal axis of the insertion portion, and is configured to be graspable by an operator; and
a flexible tube extending from a side of a major angle corresponding to the minor angle with respect to the center axis for an end portion on a proximal end side in a direction along the center axis of the grasping portion, wherein the end portion is provided at a position where a thenar portion of the operator can come into contact with the grasping portion and the flexible tube when the operator grasps the grasping portion, and an extension direction of the flexible tube is inclined to the major angle side with respect to the center axis.

2. The insertion device according to claim 1, wherein a cover member is externally mounted on an end portion on a side of the grasping portion of the flexible tube, and at least a part of an outer peripheral surface of the cover member is formed along an extension line on the major angle side of the outer peripheral portion of the grasping portion.

3. The insertion device according to claim 1, wherein an operation member is provided on the major angle side of the outer peripheral portion of the grasping portion, and the minor angle is set on an opposite side to the operation member.

4. The insertion device according to claim 1, wherein the grasping portion comprises:
a large-diameter portion formed along the center axis; and
a small-diameter portion that is juxtaposed with the large-diameter portion, has an outer diameter smaller than the large-diameter portion, and is formed so that the thenar portion can come into contact with the small-diameter portion.
